(19) United States
(12) Patent Application Publication
(10) Pub. No.: US 2006/0189908 A1
(43) Pub. Date: Aug. 24, 2006

(54) Pelvic traction harness

(76) Inventor: Jay Kennedy, Berlin, PA (US)

Correspondence Address: David J. Hill;Paul S. Weidlich, Chambliss, Bahner & Stophel, P.C., 1000 Tallan Building, Two Union Square, Chattanooga, TN, 37402, US

(21) Appl. No.: 10/355,381

(22) Filed: Jan. 31, 2003

(51) Int. Cl. A61F 5/00

(52) U.S. Cl. 602/36; 602/32

(57) ABSTRACT

A pelvic traction harness including a circumferential belt that is adapted to be secured around the torso of a patient and has a first end, a second end and a posterior area intermediate the first end and the second end. The harness also includes a separate cinching strap belt that is adapted to be secured around the circumferential belt and has a male end, a female end and an anterior region intermediate the male end and the female end. The harness also includes a pulling strap having an anterior end and a posterior end, said anterior end being adapted to be attached to anterior region of the cinching strap belt, said posterior end being adapted to be attached to the posterior area of the circumferential belt. The pulling strap is also adapted to extend between the legs of the patient and attach to a source for applying a pulling force. The invention also includes a method for applying a traction force to the spine of a patient through the pelvic region of the patient.

### FIELD OF THE INVENTION

This invention relates generally to pelvic traction harnesses used to apply a traction force to the spine of a patient through the pelvic area. More particularly, the invention relates to an apparatus and method for applying a balanced, evenly-distributed, and properly aligned traction force to the spine of a patient through the application of pulling force to the pelvic area of the patient.

### BACKGROUND OF THE INVENTION

It is known to apply a traction force to the spine of a patient by securing around the pelvic region of a patient a belt or harness having one or more pulling straps. See, e.g., U.S. Pat. Nos. 4,995,378; 4,073,290; and 3,561,434. It is further known to attach the pulling strap or straps to a pulling device. Typically, traction force is applied by a pulling device located near the feet of the patient while the patient is in a prone position. Consequently, when the pulling device is attached to the pulling strap, a pulling force is applied to the pulling strap, the belt, and the pelvic region of the patient, and a traction force is applied to the spine of the patient in the general direction of the feet of the patient. However, these conventional devices suffer from one or more disadvantages.

In general, conventional devices are secured around the torso of a patient using a single belt that relies on only the compressive or crimping force produced by the belt in order to maintain a secure, non-slipping engagement with the iliac crests of the os coxae bones of the pelvic girdle of the patient when a traction force is applied. The compressive or crimping force produced by the typical belt is generally aligned along only an axis parallel to the longitudinal axis of the single belt. In addition, conventional belts are relatively wide and distribute the compressive or crimping force along the entire width of the belt. Conventional belts also produce an uncomfortable squeezing or pinching force around the torso of the patient. These disadvantages become more pronounced in a patient having excess body mass in the area of the iliac crests.

In addition, some conventional belts must be secured around the torso of the patient before the patient assumes a prone position. Thereafter, when the patient assumes the prone position, the conventional belt is frequently not sufficiently tight to securely bear against the iliac crests of the patient when a traction force is applied. Again, these disadvantages are more pronounced when a patient has excess body mass in the area of the iliac crests.

As a result of the foregoing disadvantages, conventional devices are susceptible to movement relative to the torso of a patient in the direction of the pulling force and even losing a bearing engagement with the iliac crests of the patient when a traction force is applied. In addition, conventional devices do not produce a traction force that is optimally distributed around the torso of the patient. Further, conventional devices do not produce a traction force that is optimally aligned with the spine of the patient or optimally balanced along the posterior-anterior axis of the torso of the patient.

It would be desirable, therefore, if an apparatus and method for applying a traction force to the spine of a patient through the pelvic area could be provided that would produce a more securely bearing engagement between the apparatus and the iliac crests of a patient, thereby reducing the incidence of movement of the harness relative to the torso of the patient in the direction of a traction force applied thereto. It would also be desirable if such an apparatus and method could be provided that would produce a traction force that is more evenly and completely distributed around the torso of a patient. It would be further desirable if such an apparatus and method could be provided that would produce a traction force that is more optimally aligned with the longitudinal axis of the spine of the patient. It would be still further desirable if such an apparatus and method could be provided that would produce a traction force that is more optimally balanced along the posterior-anterior axis of the torso of the patient. It would also be desirable if such an apparatus and method could be provided that would reduce the discomfort experienced by a patient when a traction force is applied. In addition, it would be desirable if such an apparatus and method could be provided that would permit the apparatus to be secured around the torso of a patient while the patient is in a supine position. It would be further desirable if such an apparatus and method could be provided that would produce a cinching force around the torso of a patient. It would be still further desirable if such an apparatus and method could be provided that would produce a localized cinching force around the torso of a patient. It would also be desirable if such an apparatus and method could be provided that would produce a pair of distinct cinching force distributions whereby the two cinching force distributions are generally opposed to each other and directed along non-parallel planes.

### ADVANTAGES OF THE INVENTION

Accordingly, it is an advantage of the invention claimed herein to provide an apparatus and method for producing a more securely bearing engagement between the apparatus and the iliac crests of the os coxae bones of the pelvic girdle of a patient, thereby reducing the incidence of movement of the harness relative to the torso of the patient in the direction of a traction force applied thereto. It is also an advantage of the invention claimed herein to provide such an apparatus and method for producing a traction force that is more evenly and completely distributed around the torso of a patient. It is another advantage of the invention claimed herein to provide such an apparatus and method for producing a traction force that is more optimally aligned with the longitudinal axis of the spine of the patient. It is still another advantage of the invention claimed herein to provide such an apparatus and method for producing a traction force that is more optimally balanced along the posterior-anterior axis of the torso of the patient. It is yet another advantage of the invention claimed herein to provide such an apparatus and method that would reduce the discomfort experienced by a patient when a traction force is applied. It is a further advantage of the invention claimed herein to provide such an apparatus and method that would permit the apparatus to be secured around the torso of a patient while the patient is in a supine position. It is a still further advantage of the invention claimed herein to provide such an apparatus and method for producing a cinching force around the torso of a patient. It is also an advantage of the invention claimed herein to provide such an apparatus and method for producing a localized cinching force around the torso of a patient. It is another advantage of the invention claimed herein to provide such an apparatus and method for producing a pair of distinct cinching force distributions whereby the two cinching force distributions are generally opposed to each other and directed along non-parallel planes.

Additional advantages of this invention will become apparent from an examination of the drawings and the ensuing description.

### EXPLANATION OF TECHNICAL TERMS

As used herein, the term cinching effect refers to the constricting effect achieved by producing a pair of distinct, non-parallel pulling forces around the torso of a patient, whereby one of the distinct, non-parallel pulling forces is generally directed toward the anterior of the patient and the other distinct, non-parallel parallel pulling force is generally directed toward the posterior of the patient. In a preferred embodiment of the invention claimed herein, the two distinct, non-parallel pulling force distributions applied around the torso of the patient produce a V-shaped force vector configuration when viewed as a cross-section cut along the posterior-anterior plane of the patient. The cinching effect achieved by the invention claimed herein is produced, in part, by providing a pair of separate belts adapted to be secured around the torso of a patient such that each may move relative to the other. Additional factors that contribute to the cinching effect achieved by the invention claimed herein include, but are not limited to, the following: a pulling force is applied to only the anterior of the cinching strap belt; a pulling force is applied to only the posterior of the circumferential belt; the cinching strap belt is narrow; and the cinching strap belt is loosely secured to the circumferential belt such that the cinching strap belt may move relative to the circumferential belt in the general direction of the pulling force applied to the cinching strap belt.

### SUMMARY OF THE INVENTION

The invention comprises an apparatus and method for a pelvic traction harness comprising a circumferential belt, a cinching strap belt, and a pulling strap. More particularly, the apparatus of the invention includes a circumferential belt having a first end, a second end opposite the first end, a fastening means for adjustably securing the circumferential belt around the torso of the patient, and a posterior area intermediate the first end and the second end. The circumferential belt is adapted to be secured around the torso of a patient such that the posterior area of the circumferential belt is adjacent to the lower spine of the patient. In addition, a cinching strap belt having a male end, a female end, and an anterior region intermediate the male and female end. The cinching strap belt is adapted to be secured around the circumferential belt such that the anterior region of the cinching strap belt is opposite the posterior area of the circumferential belt. The invention further comprises a pulling strap having an anterior end and a posterior end. The anterior end of the pulling strap is adapted to be attached to the anterior region of the cinching strap belt, and the posterior end of the pulling strap is adapted to be attached to the posterior area of the circumferential belt. Further, the pulling strap is adapted to extend between the legs of the patient and attach to a source for applying a pulling force.

According to the method of the invention, a traction force is applied to the spine of a patient through the application of a pulling force to the pelvic region of the patient. More particularly, the method of the invention includes providing a circumferential belt having a first end, a second end opposite the first end, a posterior area intermediate the first end and the second end, a means for securing the first end to the second end, and a belt loop. The first end of the circumferential belt is secured to the second end such that the circumferential belt extends around the torso of a patient and the posterior area is adjacent to the lower spine of the patient.

In addition, the method includes providing a cinching strap belt having a male end, a female end opposite the male end, an anterior region intermediate the male end and the female end, a means for securing the male end to the female end, and a means for receiving a pulling strap in the anterior region of the cinching strap belt. The cinching strap belt is passed through the belt loop, and the male end of the cinching strap belt is secured to the female end such that the cinching strap belt extends around the circumferential belt and the anterior region of the cinching strap belt is opposite the posterior area of the circumferential belt.

The method also includes providing a pulling strap having a posterior end that is fixedly attached to the posterior area of the circumferential belt, an anterior end opposite the posterior end, a means for connecting the anterior end of the pulling strap to the anterior region of the cinching strap belt, and a means for attaching the pulling strap to a source for applying a pulling force. The means for connecting the anterior end of the pulling strap is attached to the means for receiving the pulling strap in the anterior region of the cinching strap belt such that the pulling strap extends between the legs of the patient.

The method further includes providing a source for applying a pulling force. The means for attaching the pulling strap to a source for applying a pulling force is attached to the source for applying a pulling force, and a pulling force is applied to the pulling strap.

In order to facilitate an understanding of the invention, the preferred embodiments of the invention are illustrated in the drawings, and a detailed description thereof follows. It is not intended, however, that the invention be limited to the particular embodiments described or to use in connection with the apparatus illustrated herein. Various modifications and alternative embodiments such as would ordinarily occur to one skilled in the art to which the invention relates are also contemplated and included within the scope of the invention described and claimed herein.

### BRIEF DESCRIPTION OF THE DRAWINGS

The presently preferred embodiments of the invention are illustrated in the accompanying drawings, in which like reference numerals represent like parts throughout, and in which:

FIG. 1 is a perspective drawing of a preferred embodiment of the pelvic traction harness according to the present invention showing a patient wearing the harness and lying supine.

FIG. 2 is a front view of the preferred embodiment of the pelvic traction harness shown in FIG. 1.

FIG. 3 is a rear view of the circumferential belt of the pelvic traction harness shown in FIGS. 1 and 2.

FIG. 4 is a front view of a preferred embodiment of the cinching strap belt of the pelvic harness traction that is shown in FIGS. 1 and 2.

FIG. 5 is a front view of a preferred embodiment of the pulling strap of the pelvic harness strap that is shown in FIGS. 1 and 2.

FIG. 6 is a front view of a preferred embodiment of the extension belt that is part of a preferred embodiment of the present invention.

FIG. 7 is a perspective view of the preferred embodiment of the pelvic traction harness shown in FIG. 1 being used in combination with a thoracic traction harness.

### DESCRIPTION OF THE PREFERRED EMBODIMENTS OF THE INVENTION

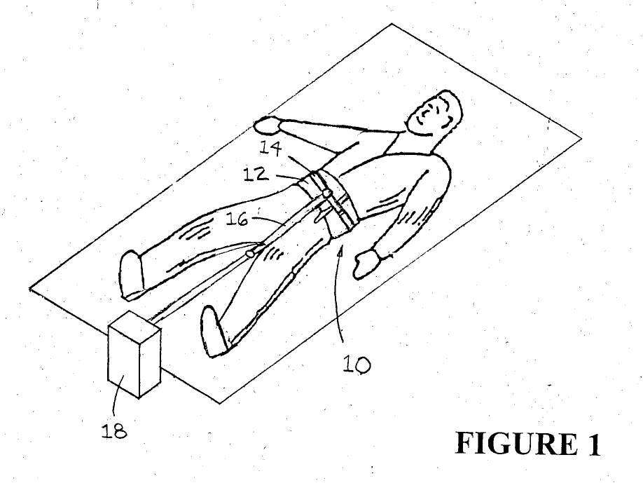

Referring now to the drawings, the apparatus and method of the invention are illustrated by FIGS. 1 through 7. FIG. 1 illustrates a preferred embodiment of the pelvic traction harness of the invention which is designated generally by reference numeral 10. More particularly, FIG. 1 illustrates a preferred embodiment of the pelvic traction harness of the invention being worn by a patient lying in the supine position.

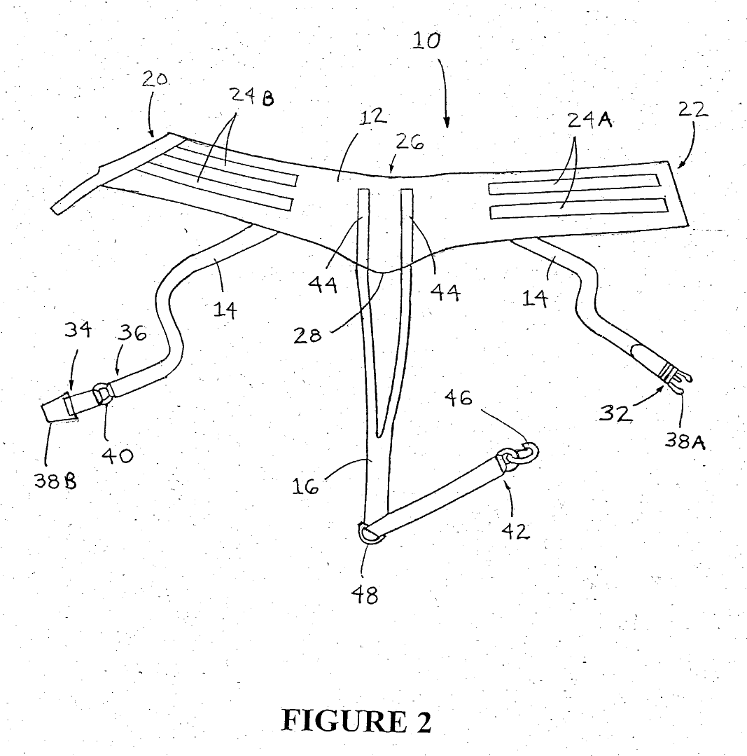

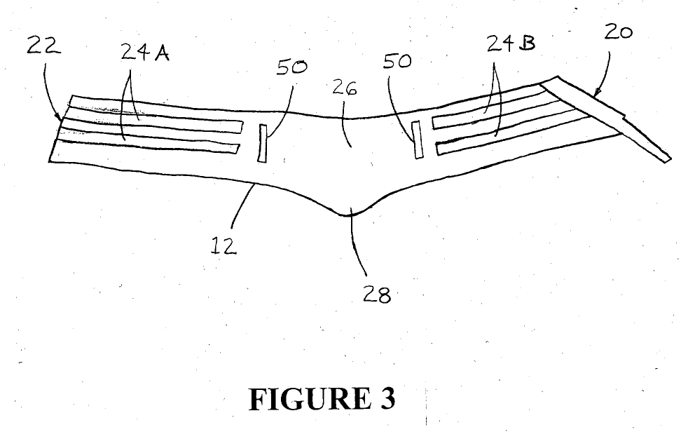

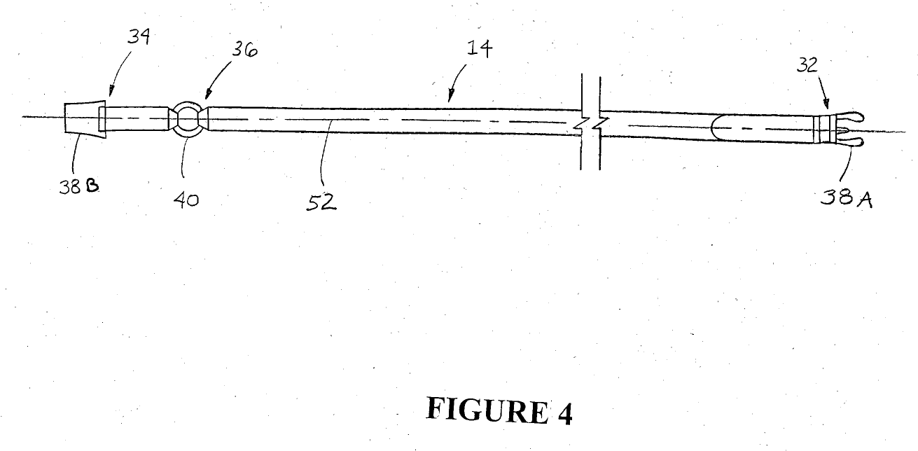

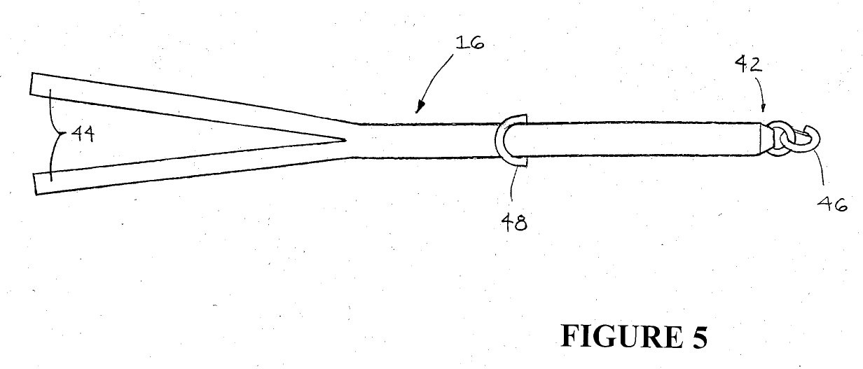

As shown in FIG. 1, pelvic traction harness 10 generally includes circumferential belt 12 (See also FIGS. 2 and 3), cinching strap belt 14 (See also FIGS. 2 and 4) and pulling strap 16 (See also FIGS. 2 and 5). Circumferential belt 12 and cinching strap belt 14 are adapted to be secured around the torso of a patient. Pulling strap 16 is adapted to be attached to the circumferential belt on one end and to the cinching strap belt on the other end such that the pulling strap extends between the legs of a patient. The pulling strap is adapted to be attached to a source for applying a pulling force such as motor 18 for the purpose of applying a traction force to the spine of the patient through bearing engagement with the pelvic girdle. More particularly, in a preferred embodiment, motor 18 applies a pulling force in the superior to anterior direction, i.e., head-to-toe, to pulling strap 16; pulling strap 16 applies a pulling force to circumferential belt 12 and cinching strap belt 14; and circumferential belt 12 and cinching strap belt 14 bear against and engage with the pelvic girdle of the patient so as to apply a traction force on the spine of the patient which effectively stretches the spinal column. It is contemplated that the source for applying the pulling force may be any suitable device for providing a traction force to harness 10. It is further contemplated within the scope of the invention that the source for applying the pulling force may be attached to pulling strap 16 by any suitable conventional means.

Referring now to FIG. 2, the preferred embodiment of pelvic traction harness 10 shown in FIG. 1 is illustrated. As shown in FIG. 2, preferred circumferential belt 12 is relatively wide as compared to cinching strap belt 14 such that it provides a large surface area in communication with the pelvic area of the patient. In a preferred embodiment, the width of circumferential belt 12 is at least 3 times the width of the cinching strap belt. Preferred circumferential belt 12 is made from a material or combination of materials that are sufficiently flexible and pliable so as to provide a comfortable, non-slipping, deformably contouring fit around the torso of a patient. In addition, the material or combination of materials used to make the circumferential belt must be sufficiently strong to withstand a pulling force applied thereto. More particularly, the preferred circumferential belt may be made from any suitable conventional material or combination of materials such as nylon, leather, cotton fabric, canvas or the like. Most preferably, the circumferential belt is made from a material or combination of materials that are adapted to comfortably, non-slippingly bear against and engage with the iliac crests of the pelvis of a patient.

As shown in FIG. 2, in a preferred embodiment, circumferential belt 12 includes first end 20 and second end 22 opposite the first end. First end 20 and second end 22 include a fastening means for adjustably securing the circumferential belt around the torso of a patient. Preferably, the fastening means is a suitable hook and loop fastener having a hook portion 24A and a loop portion 24B. However, it is contemplated within the scope of the invention that any suitable fastening means for adjustably securing the first end of the circumferential belt to the second end may be used.

Referring still to FIG. 2, preferred circumferential belt 12 includes posterior area 26 intermediate first end 20 and second end 22. In a preferred embodiment, when the circumferential belt is secured around the torso of a patient, posterior area 26 is adjacent to the lower spine or lumbosacral region of the patient.

Still referring to FIG. 2, preferred circumferential belt 12 includes projecting portion 28 in posterior area 26 of the circumferential belt (See also FIG. 3). In the preferred embodiment illustrated in the drawings, projecting portion 28 is wider than the other portions of the circumferential belt, and the posterior ends of pulling strap 16 are attached to circumferential belt 12 at or adjacent to the projecting portion.

Referring still to FIG. 2, preferred cinching strap belt 14 is relatively long and narrow as compared to the circumferential belt. The preferred cinching strap belt 14 is adapted to pass through one or more belt loops 50 on the preferred circumferential belt (See FIG. 3) such that the cinching strap belt extends around the circumferential belt. As shown in FIG. 2, preferred cinching strap belt 14 includes male end 32, female end 34, anterior region 36 intermediate the male end and the female end (See also FIG. 4), and a locking means for adjustably tightening and securing the cinching strap belt around the torso of a patient arranged on the male end and the female end. In a preferred embodiment, the locking means comprises an adjustable two-piece locking buckle having male part 38A and female part 38B (See also FIG. 4). It is contemplated, however, that any suitable conventional adjustable locking device may be used to tighten and secure the cinching strap belt around the torso of a patient. Also in a preferred embodiment, the locking means is adapted to secure the cinching strap belt such that the cinching strap belt, in combination with the circumferential belt, bears against and engages with the iliac crests of the pelvis of a patient when a pulling force is applied thereto.

As shown in FIG. 2, preferred cinching strap belt 14 also includes a means for receiving one end of pulling strap 16 (See FIG. 5) in anterior region 36 of the cinching strap belt. As shown in FIG. 2, the preferred means for receiving the anterior end of pulling strap 16 is ring 40 (See also FIG. 4). It is contemplated within the scope of the invention, however, that any suitable device for receiving the anterior end of pulling strap 16 may be used.

Still referring to FIG. 2, preferred pulling strap 16 includes anterior end 42 and a pair of posterior ends 44. Preferably, anterior end 42 is provided with a means for removably connecting the pulling strap to cinching strap belt 14 such as hook 46 (See also FIG. 5), which is adapted to be removably attached to ring 40. It is contemplated within the scope of the invention, however, that the means for removably connecting the pulling strap to the cinching belt strap may be any suitable device adapted to be used in combination with the means for receiving the anterior end of the pulling strap in order to removably attach the anterior end of the pulling strap to the cinching strap belt. Also in a preferred embodiment, the pulling strap includes a means for removably attaching the pulling strap to a source for pulling force such as D-shaped ring 48 (See also FIG. 5). In the preferred embodiment of the invention illustrated in the drawings, D-shaped ring 48 is moveable between the anterior end and the pair of posterior ends of the pulling strap. It is contemplated within the scope of the invention, however, that the means for removably attaching the pulling strap to a source for applying a pulling force such as motor 18 may be any suitable device. In addition, it is contemplated within the scope of the invention that the pulling strap may be directly attached to the source for applying a pulling force.

Referring now to FIG. 3, a rear view of the preferred embodiment of circumferential belt 12 shown in FIGS. 1 and 2 is illustrated. As shown in FIG. 3, preferred circumferential belt 12 includes a plurality of circumferential belt loops 50. Circumferential belt loops 50 are adapted to receive cinching strap belt 14. Also in a preferred embodiment, circumferential belt loops 50 are located and arranged to permit the cinching strap belt to move relative to circumferential belt 12 in the general direction of a pulling force applied to the cinching strap belt. More particularly, in a preferred embodiment, the circumferential belt loops permit the cinching strap belt to move relative to the circumferential belt along the superior-inferior axis of the patient, i.e. head-to-toe. It has been found that limiting the number of loops to 2 or 3 and minimizing the area of contact between the loops and the cinching strap belt improves the cinching effect of the harness.

Referring now to FIG. 4, the preferred embodiment of cinching strap belt 14 shown in FIGS. 1 and 2 is illustrated. As shown in FIG. 4, preferred cinching strap belt 14 is a relatively long and narrow belt such that it may extend around the circumferential belt without directly bearing against the torso of the patient. The preferred cinching strap belt is sufficiently long to be useful over a wide size range of patients. In a preferred embodiment, cinching strap belt may be as long as 65 inches or more in order to accommodate almost all patients. Also in a preferred embodiment, cinching strap belt 14 is less than about 2 inches wide so that it may provide a more localized pulling force around the torso of a patient. Consequently, the narrow cinching strap belt, in combination with the circumferential belt, more securely bears against and engages with the iliac crests of the patient. A preferred cinching strap belt 14 is made from nylon. It is contemplated, however, that cinching strap belt 14 may be made from any other suitable material having sufficient flexibility and tensile strength.

Still referring to FIG. 4, in a preferred embodiment, cinching strap belt 14 has longitudinal axis 52 and is adapted to be secured around the circumferential belt such that anterior region 36 of cinching strap belt 14 is opposite posterior area 26 of the circumferential belt. Also in a preferred embodiment, the cinching strap belt is adapted to loosely pass through belt loops 50 such that the cinching strap belt may move relative to the circumferential belt in the general direction of a pulling force applied to the cinching strap belt. Preferably, each belt loop 50 provides an opening that is at least 2 times longer than the width of the cinching strap belt. It is also preferred that the width of each belt loop is less than the width of the cinching strap belt.

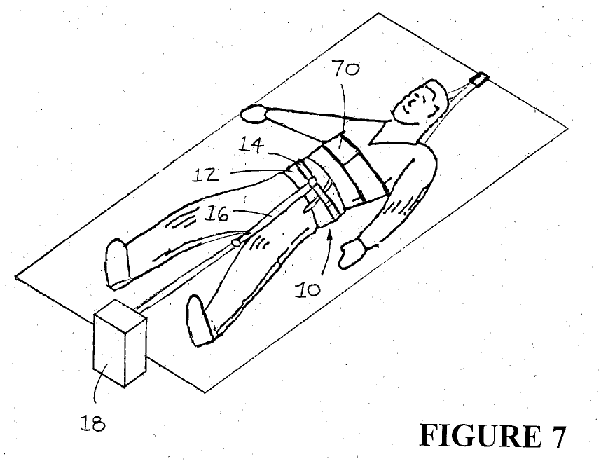

Referring now to FIG. 5, the preferred embodiment of pulling strap 16 as shown in FIGS. 1 and 2 is illustrated. Preferred pulling strap 16 is adapted to be connected on one end to circumferential belt 12 and on the other end to cinching strap belt 14 such that the pulling strap extends between the legs of a patient. (See also FIGS. 1 and 2). In addition, preferred pulling strap 16 is adapted to be attached to a source for pulling force such as motor 18 (See FIGS. 1 and 7) such that a traction force may be applied to the spine of the patient through the bearing engagement of the circumferential belt, in combination with the cinching strap belt, with the pelvic region of the patient. Pulling strap 16 may be made from nylon or any other suitable material having sufficient flexibility and tensile strength.

As shown in FIG. 5, preferred pulling strap 16 is Y-shaped. More particularly, preferred pulling strap 16 has anterior end 42 and a pair of posterior ends 44 opposite the anterior end (See also FIG. 2). While FIG. 5 illustrates pulling strap 16 with a pair of posterior ends 44, it is contemplated within the scope of the invention that more or less than two posterior ends may be provided. Further, although FIG. 5 illustrates pulling strap 16 having only one anterior end 42, it is contemplated within the scope of the invention that more than one anterior end may be provided. Finally, although FIG. 5 illustrates a single, integral pulling strap 16, it is contemplated within the scope of the invention that pulling strap 16 may comprise two or more pulling straps.

Still referring to FIG. 5, in a preferred embodiment, anterior end 42 of pulling strap 16 is adapted to be removably attached to anterior region 36 of cinching belt strap 14, and posterior ends 44 of pulling strap 16 are fixedly attached to posterior area 26 of circumferential belt 12. Also in a preferred embodiment, circumferential belt 12 is placed on the patient's torso in such a position that each of the pair of posterior ends 44 is affixed to the circumferential belt at an equal distance from the spinal column of the patient. Alternatively, a pulling strap having a single posterior end may be affixed to the circumferential belt directly in line with the spinal column of the patient. In another alternative embodiment, three posterior ends may be affixed to the circumferential belt such that the two outer posterior ends are spaced an equal distance from the patient's spinal column and the inner posterior end is directly in line with the patient's spinal column. It is also contemplated that more than three posterior ends of the pulling strap may be affixed to the posterior area of the circumferential belt in a similar balanced spaced relationship relative to the spinal column of the patient. Similarly, it is contemplated that one or more anterior ends of the pulling strap may be removably attached to the anterior region of the cinching strap belt such that the one or more anterior ends are in a balanced spaced relationship relative to the spinal column of the patient as described above with respect to the posterior ends.

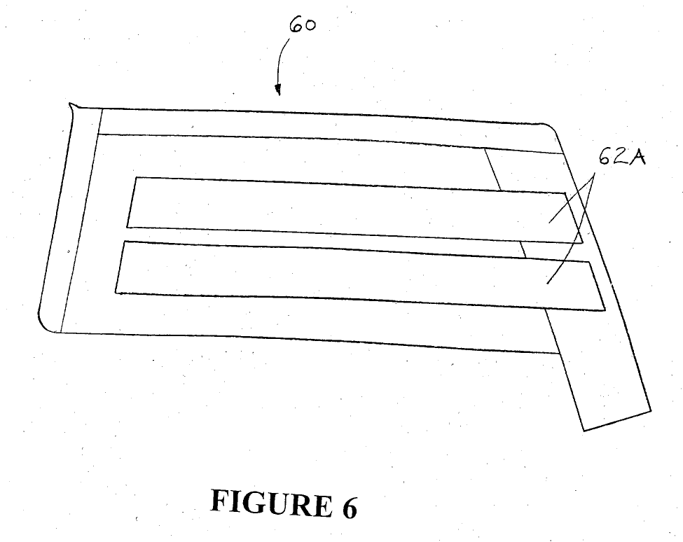

Referring now to FIG. 6, a preferred embodiment of extension belt 60 is illustrated. Preferred extension belt 60 is adapted to be removably secured to circumferential belt 12 so as to increase the length of the circumferential belt. More particularly, extension belt 60 is sufficiently long to be useful, in combination with the circumferential belt, over a wide size range of patients. In a preferred embodiment, extension belt 60 is approximately 19 inches long. When the preferred extension belt is used in combination with a preferred circumferential belt having a length of approximately 55 inches, the combination can be used with almost all patients.

As shown in FIG. 6, extension belt 60 includes means for removably securing the extension belt to the circumferential belt such as a hook and loop fastener. More particularly, the preferred extension belt includes extension belt hook portion 62A on one side and extension belt loop portion 62B (not shown) on the opposite side. As a result, preferred extension belt 60 may be attached to the first end of the circumferential belt by contacting it with extension belt hook portion 62A. The preferred extension belt 60 may then be attached to the second end of the circumferential belt by contacting it with extension belt loop portion 62B. When used in combination, the extension belt, the circumferential belt and the long and narrow cinching strap belt are adapted to accommodate patients of nearly any size or shape. This combination results in several advantages, including elimination of the need to manufacture, purchase, use and store multiple different-sized pelvic traction harnesses.

Referring now to FIG. 7, the preferred embodiment of the pelvic traction harness shown in FIGS. 1 and 2 is shown being used in combination with thoracic traction harness 70. Thoracic traction harness 70 is adapted to be secured around the upper torso of a patient. The preferred thoracic traction harness is also adapted to be secured to a stationary object such as the table on which the patient is lying so as to provide resistance to movement of the patient in the direction of the pulling force applied by the pelvic traction harness. It is contemplated within the scope of the invention that any suitable conventional thoracic traction harness may be used in combination with the pelvic traction harness of the invention.

The invention described and claimed herein also comprises a method for applying a traction force to the spine of a patient through the application of a pulling force to the pelvic region of the patient. According to the preferred method of the invention, a circumferential belt such as circumferential belt 12 described above and illustrated in FIGS. 1 through 3 is provided. More particularly, a circumferential belt having a first end, a second end opposite the first end, a means for securing the first end to the second end, a posterior area intermediate the first end and the second end, and a circumferential belt loop is provided. The first end of the circumferential belt is secured to the second end such that the circumferential belt extends around the torso of a patient and the posterior area is adjacent to the lower spine of the patient. According to a preferred embodiment of the invention, the first end of the circumferential belt may be secured to the second end such that the circumferential belt extends around the torso of a patient while the patient is in a supine position. Also according to a preferred embodiment of the invention, an extension belt such as extension belt 60 described above and illustrated in FIG. 6 may be secured to the circumferential belt so as to increase the effective length of the circumferential belt.

Also according the preferred method of the invention, a cinching strap belt such as cinching strap belt 14 described above and illustrated in FIGS. 1, 2 and 4 is also provided. More particularly, a cinching strap belt having a male end, a female end opposite the male end, a means for securing the male end to the female end, an anterior region intermediate the male end and the female end, and a means for receiving a pulling strap in the anterior region of the cinching strap belt is provided. The cinching strap belt is passed through the one or more circumferential belt loops. The male end of the cinching strap belt is secured to the female end such that the cinching strap belt preferably extends around the circumferential belt and the anterior region of the cinching strap belt is opposite the posterior area of the circumferential belt. Also in a preferred embodiment, the male end of the cinching strap belt may be secured to the female end while the patient is in a supine position.

Also according to the method of the invention, a pulling strap such as pulling strap 16 described above and illustrated in FIGS. 1 and 5 is provided. More particularly, a pulling strap having a posterior end that is fixedly attached to the posterior area of the circumferential belt, an anterior end opposite the posterior end, a means for connecting the anterior end of the pulling strap to the anterior region of the cinching strap belt, and a means for attaching the pulling strap to a source for pulling force is provided. The means for connecting the anterior end of the pulling strap to the anterior region of the cinching strap belt is attached to the means for receiving the pulling strap in the anterior region of the cinching strap such that the pulling strap extends between the legs of the patient.

Also according to the method of the invention, a source for pulling force such as motor 18 shown in FIGS. 1 and 7 is provided. The means for attaching the pulling strap to a source for pulling force is connected to the source for pulling force, and a pulling force is applied to the pulling strap. Preferably, the source for pulling force is located in the area of the patient's feet so as to apply a pulling force in a direction that urges the pelvic region of the patient toward his or her feet, thereby stretching the spine of the patient.

In operation, the several advantages of the apparatus and method of the invention are achieved. More particularly, the apparatus and method of invention produce a non-slipping, bearing engagement between the apparatus and the iliac crests of a patient, thereby reducing the incidence of movement of the harness relative to the torso of the patient in the direction of a pulling force applied thereto. The invention achieves this non-slipping, bearing engagement between the harness and the iliac crests of the patient as a result of a number of aspects of the invention. For example, the invention claimed herein produces a traction force that is more evenly and completely distributed around the torso of a patient. The invention claimed herein also produces a traction force that is more optimally aligned with the longitudinal axis of the spine of the patient. Further, the invention claimed herein produces a traction force that is more optimally balanced along the posterior-anterior axis of the torso of a patient. Still further, the invention claimed herein also reduces the discomfort experienced by a patient when a traction force is applied thereto. In addition, the invention claimed herein permits the apparatus of the invention to be secured around the torso of a patient while the patient is in a supine position.

The non-slipping, bearing engagement between the apparatus of the invention and the iliac crests of the patient is further enhanced because the invention claimed herein produces a cinching effect around the torso of a patient. The cinching effect produced by the apparatus and method of the invention results from a number of aspects of the invention. For example, the cinching strap belt is loosely secured around the circumferential belt by the circumferential belt loop or loops such that the cinching strap belt may move relative to the circumferential belt in the general direction of the pulling force applied to the cinching strap belt. Further, the anterior end of the pulling strap is attached to only the anterior region of the cinching strap belt, and therefore, it urges the cinching strap belt toward the anterior of the patient. In addition, the cinching effect of the harness is further enhanced because the posterior ends of the pulling strap are attached to only the posterior area of the circumferential belt. Consequently, the circumferential belt is urged toward the posterior of the patient. The combination of the anterior-urging cinching strap belt and the posterior-urging circumferential belt produces a cinching effect that results in an improved non-slipping, bearing engagement between the apparatus of the invention and the pelvic region of the patient.

In addition, the combination of the two distinct and generally opposed pulling forces applied by the cinching strap belt and the circumferential belt further improves the cinching effect of the harness because the two generally opposed pulling forces may be distributed along non-parallel planes. More particularly, the two distinct belts may produce a pair of distinct, non-parallel pulling forces that form a generally V-shaped pull vector configuration. By contrast, conventional harnesses using only a single, integral belt cannot produce a pair of distinct, generally opposed, non-parallel pull vectors that will have a cinching effect like the invention claimed herein. Instead, conventional single-belt harnesses, even those with a pulling strap attached on one end to the anterior of the belt and on the other end to the posterior of the belt, must rely on only the compressive or crimping force of the belt in order to resist movement relative to the torso of a patient in the direction of a pulling force. The cinching effect of the harness is further enhanced because the cinching strap belt is relatively narrow, and therefore, it applies a pulling force that may be localized to the area just above the iliac crests of the patient.

It has also been found that the pelvic traction harness of the invention may be advantageously utilized in combination with a thoracic traction harness adapted to be secured around the upper torso of a patient. The preferred thoracic traction harness is also adapted to be secured to a stationary object such as the table on which the patient is lying such that the thoracic traction harness provides resistance to movement of the patient in the direction of the pulling force applied by the pelvic traction harness.

Although this description contains many specifics, these should not be construed as limiting the scope of the invention but as merely providing illustrations of some of the presently preferred embodiments thereof, as well as the best mode contemplated by the inventor of carrying out the invention. The invention, as described herein, is susceptible to various modifications and adaptations, and the same are intended to be comprehended within the meaning and range of equivalents of the appended claims.

What is claimed is:

1. A pelvic traction harness comprising:
(a) a circumferential belt having a first end, a second end opposite the first end, a fastening means for adjustably securing the circumferential belt around the torso of the patient, a posterior area intermediate the first end and the second end, and a circumferential belt loop said circumferential belt being adapted to be secured around the torso of a patient such that the posterior area of the circumferential belt is adjacent to the lower spine of the patient;
(b) a cinching strap belt having a male end, a female end opposite the male end, and an anterior region intermediate the male end and the female end, said cinching strap belt being adapted to be removably secured around the circumferential belt by passing the cinching strap belt through the circumferential belt loop such that the anterior region of the cinching strap belt is opposite the posterior area of the circumferential belt; and,
(c) a pulling strap having an anterior end and a posterior end, said anterior end being adapted to be attached to the anterior region of the cinching strap belt, said posterior end being adapted to be attached to the posterior area of the circumferential belt, and said pulling strap being adapted to extend between the legs of the patient and adapted to be attached to a source for applying a pulling force.

2. The harness of claim 1 wherein the circumferential belt includes a plurality of loops through which the cinching strap belt may be passed.

3. The harness of claim 2 wherein the circumferential belt loops are adapted to permit the cinching strap belt to move relative to the circumferential belt in the general direction of a pulling force applied to the cinching strap belt.

4. The harness of claim 1 wherein the fastening means for adjustably securing the circumferential belt around the torso of the patient is a hook and loop fastener arranged to secure the first end of the circumferential belt to the second end of the circumferential belt.

5. The harness of claim 1 wherein the circumferential belt includes a projecting portion in the posterior area of the circumferential belt, wherein said projecting portion is wider than the other portions of the circumferential belt.

6. The harness of claim 1 wherein the pulling strap is attached to the projecting portion of the circumferential belt.

7. The harness of claim 1 wherein the cinching strap belt includes a locking means for adjustably tightening and securing the cinching strap belt around the torso of the patient.

8. The harness of claim 7 wherein the locking means is adapted to cinch the cinching strap belt around the circumferential belt such that the harness bears against and engages with the iliac crests of the patient when a pulling force is applied to the pulling straps.

9. The harness of claim 8 wherein the locking means for adjustably tightening and securing the cinching strap belt around the circumferential belt is a locking buckle.

10. The harness of claim 1 wherein the cinching strap belt has a width that is less than about one-third of the width of the circumferential belt.

11. The harness of claim 1 wherein the anterior end of the pulling strap includes a means for removably connecting the pulling strap to the cinching strap belt.

12. The harness of claim 1 wherein the posterior end of the pulling strap is fixedly attached to the posterior area of the circumferential belt.

13. The harness of claim 1 wherein the pulling strap includes a means for removably attaching the pulling strap to a source for applying a pulling force.

14. The harness of claim 1 wherein the pulling strap includes a plurality of posterior ends that are fixedly attached to the posterior area of the circumferential belt.

15. The harness of claim 1 which includes an extension belt that is adapted to be removably secured to the circumferential belt so as to increase the length of the circumferential belt.

16. The harness of claim 1 wherein the circumferential belt applies a first pulling force to the pelvic region of a patient and the cinching strap belt applies a second pulling force to the pelvic region of the patient, said first pulling force and said second pulling force being generally opposed and non-parallel relative to each other.

17. A pelvic traction harness for applying a traction force to the spine of a patient through the pelvic region of the patient, said harness comprising:
(a) a circumferential belt comprising:
(i) a first end and a second end opposite said first end;
(ii) an anterior area and a posterior area opposite said anterior area;
(iii) a belt loop;
wherein said circumferential belt is adapted to be secured around the torso of the patient such that the anterior area of the circumferential belt is adjacent to the lower abdomen of the patient and the posterior area of the circumferential belt is adjacent to the lower spine of the patient;
(b) a cinching strap belt comprising:
(i) a male end and a female end opposite said male end;
(ii) an anterior region and a posterior region opposite said anterior region;
(iii) a cinching strap belt ring in said anterior region;
wherein said cinching strap belt is adapted to removably pass through the belt loop and tighten around the circumferential belt using a locking buckle arranged on the male end and the female end of the cinching strap belt such that the anterior region of the cinching strap belt is adjacent to the anterior area of the circumferential belt and the posterior region of the cinching strap belt is adjacent to the posterior area of the circumferential belt;
(c) a Y-shaped pulling strap comprising:
(i) an anterior end and a pair of posterior ends opposite said anterior end;
(ii) a hook on the anterior end;
(iii) a pulling strap ring that is movable between the anterior end and the pair of posterior ends;
wherein the hook on the anterior end of the Y-shaped pulling strap is adapted to be removably attached to the ring on the cinching strap belt, the posterior ends of the Y-shaped pulling strap are fixedly attached to the posterior area of the circumferential belt, the Y-shaped pulling strap is adapted to extend between the legs of the patient, and the pulling strap ring is adapted to be removably attached to a source for applying a pulling force;
whereby the harness produces a pair of distinct, non-parallel, generally opposed pulling forces adapted to produce a cinching effect around the torso of the patient.

18. A method for applying a traction force to the spine of a patient through the pelvic region of the patient, said method comprising the steps of:
(a) providing a circumferential belt, said circumferential belt having a first end, a second end opposite said first end, a posterior area intermediate the first end and the second end, a means for securing the first end to the second end, and a belt loop;
(b) securing the first end of the circumferential belt to the second end such that the circumferential belt extends around the torso of a patient and the posterior area is adjacent to the lower spine of the patient;
(c) providing a cinching strap belt, said cinching strap belt having a male end, a female end opposite the male end, an anterior region intermediate the male end and the female end, a means for securing the male end to the female end, and a means for receiving a pulling strap in the anterior region of the cinching strap belt;
(d) passing the cinching strap belt through the belt loop;
(e) securing the male end of the cinching strap belt to the female end such that the cinching strap belt removably extends around the circumferential belt and the anterior region of the cinching strap belt is opposite the posterior area of the circumferential belt;
(f) providing a pulling strap, said pulling strap having a posterior end that is fixedly attached to the posterior area of the circumferential belt, an anterior end opposite the posterior end, a means for connecting the anterior end of the pulling strap to the anterior region of the cinching strap belt, and a means for attaching the pulling strap to a source for applying a pulling force;
(g) attaching the means for connecting the anterior end of the pulling strap to the means for receiving the pulling strap in the anterior region of the cinching strap belt such that the pulling strap extends between the legs of the patient;
(h) providing a source for applying a pulling force;
(i) connecting the means for attaching the pulling strap to a source for applying a pulling force to the source for applying a pulling force; and,
(j) applying a pulling force to the pulling strap.

19. The method of claim 18 wherein the step of securing the first end of the circumferential belt to the second end such that the circumferential belt extends around the torso of the patient and the step of securing the male end of the cinching strap belt to the female end such that the cinching strap belt extends around the circumferential belt are performed while the patient is in a supine position.

20. The method of claim 18 further including the steps of providing a thoracic traction harness adapted to be secured around the upper torso of a patient and attached to an object that will provide resistance to a pulling force applied to the pelvic traction harness, securing the thoracic traction harness around the upper torso of the patient, and attaching the thoracic traction harness to the object that will provide resistance to the pulling force applied to the pelvic traction harness, wherein each of said steps are performed before applying a pulling force to the pulling strap.
